(19) United States
(12) Patent Application Publication
(10) Pub. No.: US 2005/0186290 A1
(43) Pub. Date: Aug. 25, 2005

(54) Use of aquaglyceroporin modulators as slimming agent

(75) Inventor: Marie-Madeleine Cals-Grierson, Meudon (FR)

Correspondence Address: LERNER, DAVID, LITTENBERG,;KRUMHOLZ & MENTLIK, 600 SOUTH AVENUE WEST, WESTFIELD, NJ, 07090, US

(73) Assignee: L'OREAL, Paris (FR)

(21) Appl. No.: 11/009,483

(22) Filed: Dec. 10, 2004

Related U.S. Application Data
(60) Provisional application No. 60/538,345, filed on Jan. 22, 2004.

(30) Foreign Application Priority Data
Dec. 10, 2003 (FR) 0351015

(51) Int. Cl. A61K 35/78

(52) U.S. Cl. 424/725; 424/728; 424/733; 424/765; 424/746; 424/776; 424/766; 424/769; 424/727; 424/730

(57) ABSTRACT

The invention relates to the use of at least one modulator of aquaporin adipose (AQPap) in a cosmetic composition as slimming agent for reducing the volume of adipocytes and for enhancing the departure of glycerol from the adipocytes. It also relates to cosmetic compositions comprising a combination of at least one agent which modulates aquaporin adipose and of at least one lipolytic active agent.

### CROSS REFERENCE TO RELATED APPLICATIONS

In accordance with 35 U.S.C. § 119(a), this application claims the benefit of the filing date of French Application No. FR 0351015 filed Dec. 10, 2003 and in accordance with 35 U.S.C. § 119(e), this application also claims the benefit of the filing date of U.S. Provisional Patent Application No. 60/538,345 filed Jan. 22, 2004, the disclosures of which are hereby incorporated herein by reference.

### BACKGROUND OF THE INVENTION

The present invention relates to the use of aquaglyceroporin modulators as slimming agents and to cosmetic compositions comprising such modulators.

Plumpness and/or excess weight are related to the dysfunctioning of certain cells of the hypodermis, known as adipocytes, which comprise variable amounts of fats stored in the form of triglycerides. These triglycerides are synthesized in vivo by the adipocytes themselves, according to reactions of enzymatic type (lipogenesis), from the free fatty acids present in the blood in the form of lipoproteins and from the glucose provided in particular by certain foods. The release of the fatty acids from the lipoproteins takes place with the help of an enzyme, lipoprotein lipase, present in the adipocytes or via cell receptors for HDL, LDL or VLDL lipoproteins. The conversion of the glucose results either in the formation of glycerol or in the formation of free fatty acids via a specific enzyme, acetyl-CoA carboxylase, which converts the glucose to acetyl-CoA and then to fatty acids.

In point of fact, at the same time, the triglycerides thus formed and then stored in the adipocyte cells can also decompose (lipolysis), still under the action of specific enzymes, triglyceride lipases, which are present in these same cells and which are capable of being activated by cyclic AMP. Cyclic AMP is regulated by adenylate cyclase and is capable of being hydrolysed to 5′AMP by phosphodiesterase. This lipolysis mechanism results in the release of fatty acids, on the one hand, and of glycerol and/or of glycerol mono- and/or diesters, on the other hand.

The fatty acids thus released can then either diffuse into the body, to be consumed or converted therein in various ways, or can be recaptured (immediately or slightly later) by the adipocytes so as to regenerate triglycerides by lipogenesis.

If, for various reasons (excessively rich or unbalanced diet, lack of exercise, variation in metabolism, ageing and others), a substantial imbalance is established in the body between lipogenesis (formation of triglycerides by enzymatic reaction between fatty acids and glycerol) and lipolysis (enzymatic decomposition of triglycerides to give fatty acids and glycerol), that is to say, more specifically, if the amounts of fat formed by lipogenesis become significantly and continually greater than those which are removed by lipolysis, then accumulation of triglycerides occurs in the adipocytes, which, if it becomes excessive, may be gradually reflected by deformation of the skin brought about by the thickening of the hypodermis in which the adipocytes are found. The surface of the skin becomes uneven (orange peel) and to a greater or lesser extent has a flabby or jelly-like consistency, finally giving the figure an unsightly general appearance which can progress from simple local accumulation of fat (lipodysmorphia), to definite stoutness, and finally real obesity.

In point of fact, in view in particular of the great unpleasantness, both physical and aesthetic, and sometimes psychological, which is caused in individuals who are affected by it, adiposity is nowadays a disorder which is increasingly poorly endured or accepted.

Solutions have been provided in the prior art for intervening in the metabolism of the fatty acids, which is one of the favoured targets in the control of this adipocytic lipid accumulation.

This metabolism can be adjusted:
either by blocking the transportation of the glucose inside the adipocyte, which results in a reduction in the fatty acids entering the adipocyte,
or by inhibition of lipoprotein lipase,
or by activation of triglyceride lipase (or hormone-sensitive lipase), generally by stimulating cyclic AMP, generally by activation of adenylate cyclase, or by causing it to accumulate by inhibition of phosphodiesterase.

Other biological routes have been explored for acting on the mechanism of lipogenesis and/or of lipolysis. Thus, the proposal has been made to use neuropeptide Y (NPY) receptor antagonists, neuropeptide Y being a neuromediator involved in a number of physiological processes, the involvement of which in the regulation of lipolysis it has been possible to demonstrate (P. Valet, J. Clin. Invest., 1990, 85, 291-295). Use may also be made of α₂ receptor antagonists or β₃-adrenergic receptor agonists.

The cosmetic compositions provided to date for the purpose of treating adiposity thus comprise compounds, referred to as slimming compounds, which act on one or more of the mechanisms mentioned above. Among these, mention may more particularly be made of xanthine bases (i.e. xanthine derivatives), such as theophylline, caffeine, theobromine and 1-hydroxyalkylxanthines and their compatible salts (see the document FR-A-2 617 401), which are phosphodiesterase inhibitors, nicotinic acid derivatives, such as more particularly α-tocopherol nicotinate and hexyl nicotinate (see the document EP-A-371 844), substances referred to as α₂ blockers, capable of blocking α₂ receptors at the surface of the adipocytes, such as, for example ginkgo biloba (see Patents FR-A-2,669,537 or U.S. Pat. No. 5,194,259), and β₃-adrenergic receptor agonists, such as alverine and its salts.

Application WO 00/30603 discloses slimming compositions comprising Dioscorea opposita extract. The document FR 2,819,409 also relates to the use of diosgenin, which can be obtained by synthesis or extracted from Dioscorea opposita, in slimming compositions, this compound reducing the accumulation of triglycerides in the adipocyte by competitiveness.

U.S. Pat. No. 6,399,089 discloses compositions for regulating the body weight by inhibition of the recapture of serotonin and inhibition of lipogenesis, it being possible for these compositions to comprise quercetin, cocoa, chromium and Hypericum perforatum, Garcinia cambodgia, Ginkgo biloba and Panax ginseng extracts, all of these compounds having to be present.

WO 01/64177 relates to the use, in the treatment of cellulite, of flavones, isoflavones or their glycosides; these compounds are introduced in particular in the form of soybean extracts. FR 2,578,165 also discloses the use of bioflavonoids, in combination with Hedra helix and Fucus vesiculorum tinctures, in anticellulite ointments.

FR 2,774,589 provides a mixture of mucopolysaccharidase and aescin in slimming cosmetic compositions; the role of the aescin is to stimulate lipolysis by masking the β-adrenergic receptors of the adipocyte.

WO 02/089758 discloses the use of procyanidol oligomers (PCOs), in particular Vitis vinfera extracts, in skincare compositions with an anti-ageing purpose.

FR 2,790,645 relates to food supplements comprising a grape extract rich in polyphenols, targeted at inhibiting emulsification of lipids in the gastric environment.

WO 92/15293 relates to the treatment or to the prevention of cellulite by retinoids.

U.S. 2002/0106388 relates to methods for the treatment of cellulite by formulations which make it possible to block oestrogen receptors, to inhibit destruction of collagen and to stimulate lipolysis; the compositions comprise a mixture of isoflavones, such as genistein or quercetin, of carnitine derivatives, of xanthine bases, such as theophylline, and of Coleus forskolii extract.

These various solutions can given good results but there still exists a need to have available novel means for combating unattractive excess weight, in particular local lipodystrophia, for the purpose of obtaining a general effect or, on the contrary, a local effect of slimming and/or refining the body or the face.

In this respect, the availability of the precursors of lipogenesis in adipocytes is an important regulatory factor. This is because it is necessary to prevent the metabolites generated by the lipolysis from being immediately reused by the adipocyte to store triglycerides.

### SUMMARY OF THE INVENTION

In the context of the present invention, it has now been found that it is possible to act by promoting the removal of one of these precursors by the adipocyte.

For this reason, a subject-matter of the present invention is the use of at least one aquaglyceroporin adipose (AQPap) modulator in a cosmetic composition as slimming agent for reducing the volume of adipocytes.

Aquaporins are membrane (channel) proteins which bring about the passage of water and small molecules, such as urea and glycerol, and which belong to the MIP (major intrinsic protein) family. They have been identified on cells, both in various organs in mammals and in plants or prokaryotic organisms. Two subgroups are functionally distinguished: aquaporins, which are selective for water or urea, and aquaglyceroporins, which also transport glycerol.

Approximately 10 types of aquaporins (also referred to as AQP subsequently) have been identified in the tissues of mammals, such as AQP1 in erythrocytes, AQP2, AQP3 and AQP6 in the kidney, AQP4 in the brain, AQP5 in the lachrymal and salivary glands, and AQP9 in the liver. A specific aquaporin of the adipose tissue was characterized by Kuriyama et al. (Biochem. Biophys. Res. Commun., 1997, 241, 53-58): it is an aquaglyceroporin, known as AQPap, and it exhibits similarities with AQP7 of the rat testis. The expression of the AQPap gene is repressed by insulin (Kishida et al., 2001, J. Biol. Chem., 276, 36251-36260).

The use of aquaporins or aquaporin modulators has been proposed in cosmetics for promoting moisturizing of the skin, for example in FR 2 831 058; Application WO 01/37799 provides for Ajuga turkenistanica extracts for regulating aquaporins 3 and provides better moisturizing of the epidermis. Furthermore, Application EP 1,084,700 discloses antiperspirant compositions comprising an effective amount of an aquaporin modulator.

However, to date, provision has never been made to use aquaporin modulators as slimming agent.

In agreement with the invention, AQPap modulators reduce the volume of the adipocytes, on the one hand by limiting the immediate resynthesis of triglycerides from the glycerol released and, on the other hand, by reducing the content of glycerol and of water in these cells. The term “AQPap modulators” according to the invention is preferably understood to mean an agent capable of stimulating the AQPap activity of the adipocytes, specifically of the mature adipocytes. Advantageously, the AQPap modulator enhances the departure of glycerol from the adipocytes.

### DETAILED DESCRIPTION OF THE INVENTION

According to one of the aspects of the invention, the AQPap modulators enhance the synthesis of aquaglyceroporin by the adipocytes. According to another of its aspects, the modulator enhances the departure of glycerol from the adipocytes by promoting the opening of the AQPap channels, by increasing their number and/or by promoting their translocation towards the membrane, or via a combination of these mechanisms.

AQPap modulators of use according to the invention can be chosen in particular from the group consisting of polyphenols, procyanidins (B2 or C1, for example), catechins in the monomer and/or polymer forms, anthocyanidins, proanthocyanidins, flavonoids, phyto-oestrogens, quercetin, plant extracts comprising the same, retinoids and plant extracts chosen from ginseng, sage, chocolate, cocoa, apples, particularly the Red Delicious and Granny Smith varieties, red wine, bilberries, blueberry, the herb Hypericum perforatum, Hamamelis, Ceratonia siliqua (locust bean), Musanga cecropioides, leaves and flowers of Crataegus laevigata (hawthorn), coconut (Cocos nucifer) fibres, Sorgum bicolour, Pyrus communis flowers, kiwi fruit, pycnogenol, tomato, aubergine, Guazuma ulmifolia, rhubarb, Uncaria sp., and their mixtures.

The AQPap modulator can be introduced into the composition in the purified form, in particular by a synthetic molecule, or in the form of an extract, for example a plant or bacterial extract, comprising one or more AQPap-modulating agents, optionally in combination with other components.

In particular, the modulator can be chosen from cocoa powder extracts with a determined content of polyphenols and of proanthocyanidin oligomers, such as the product Caophenol® sold by Solabia, aescin, horse chestnut proanthocyanidins, complexes of phospholipids and of horse chestnut bark proanthocyanidins, in particular the product sold as an anti-ageing product under the name PA2 Affilene® by Idena, sapogenins, such as diosgenin and in particular Dioscorea opposita (or yam) tuber extracts with a determined content of diosgenin sold under the name Dioschol® by Sederma, sage extracts and in particular fermented sage extracts, (tridec-2-ynylthio)acetic acid and {[4-(octylthio)but-2-ynyl]thio}acetic acid.

Preferably, the modulators of use according to the invention induce, when they are introduced into a culture of mature adipocytes, an increase in the synthesis of AQPap, in particular of the AQPap gene, by these adipocytes of greater than or equal to 1.5 times, with respect to a culture of mature adipocytes in the absence of AQPap modulator.

AQPap modulators suited to the implementation of the invention can thus be selected by a process comprising the following stages:
a) cultures of differentiated mature adipocytes are prepared (i) in the presence of the test product and (ii) in the absence of the test product,
b) after incubation, the cells cultured according to (i) or (ii) are harvested,
c) the total RNA is extracted and the expression of the gene corresponding to the AQPap in the cells cultured in the presence (i) or in the absence (ii) of the test product is compared,
d) the product for which the expression of the AQPap in the adipocytes cultured in its presence is increased by a factor of at least 1.5 with respect to the culturing carried out without the test product is selected.

Advantageously, the comparison of the expression of the gene in the two types of culturing, in stage c), is carried out by techniques employing quantitative RT-PCR.

Alternatively, an AQPap modulator according to the invention is selected by a process in which stages a) and b) are similar to the above and the amount of the AQPap protein present in the cells is subsequently evaluated, either after extraction, by labelling by techniques known to a person skilled in the art, such as the Western blotting, or by visualization on the fixed culture, for example by immunofluorescence. As above, the products for which the amount of AQPap in the adipocytes originating from the cultures (i) in the presence of the product is greater than or equal to 1.5 times the amount of AQPap in the cultures (ii) will be selected.

The modulators selected by the process according to the invention correspond to agents which increase the number of AQPap channels.

Other AQPap-modulating active agents of use according to the invention can be demonstrated by a process comprising the stages consisting in:
culturing mature differentiated adipocytes in the presence or in the absence of the test product,
comparing the amount of glycerol present in the culture supernatant after incubation in the cultures produced in the presence of the test product to the amount of glycerol present in the culture supernatant after incubation without addition of the test product,
selecting the product for which the amount of glycerol released into the culture supernatant of the adipocytes is increased by at least 1.2 times and in particular by at least 1.5 times with respect to the amount of glycerol in the culture supernatant of mature adipocytes which have not received AQPap modulator.

The products thus selected are AQPap activators which can be used according to the invention.

These AQPap modulators bring about the reduction in the volume of the adipocytes, and thus of the fatty tissue, by promoting the removal of the glycerol originating in particular from the physiological lipolysis. Furthermore, they may additionally have an intrinsic lipolytic activity and thus a slimming activity which combines different mechanisms.

According to one of the aspects of the invention, the AQPap modulator also promotes the departure of the non-esterified free fatty acids (or NEFAs) from the adipocyte. From lipolysis, and thus cleavage of triglycerides into NEFA and glycerol, for some modulators, when the functioning of the AQPap channel is inhibited, for example by the presence of a mercury salt, it is observed that the glycerol no longer leaves the adipocyte cell, demonstrating that the release of glycerol involves the AQPap channels; on the other hand, the NEFAs are still excreted by the cell, via a route other than the AQPaps.

According to an advantageous embodiment of the invention, a combination of at least one modulator capable of increasing the number of the AQPap channels and at least one AQPap modulator capable of stimulating the intrinsic activity of the AQPap channels is used.

The term “agent which stimulates the intrinsic activity of the AQPap channels” is understood to mean an agent which promotes the translocation of the AQPap channels towards the membrane of the adipocyte and/or which promotes the opening of the said AQPap channels (and thus the passage of the glycerol through the AQPap channels).

The agent capable of increasing the number of the AQPap channels is in particular an agent capable of enhancing the expression and/or the synthesis of the AQPap.

According to one of the aspects of the invention, the same agent fulfils the two functions by increasing the number of the AQPap channels and by stimulating their intrinsic activity.

According to another of its aspects, a combination of at least two different modulators is used, the functions of which at the level of the activity with regard to the aquaporin channels will be at least partially complementary.

In particular, in such combinations, the modulating agent capable of increasing the activity of aquaporin adipose (AQPap) channels can be chosen from the group consisting of Dioscorea opposita sapogenins, such as diosgenin and in particular Dioschol®, lycopene encapsulated in the form of nanocapsules, in particular of polycaprolactone comprising a tomato extract of 0.5% enriched in lycopene, and cocoa powder extracts with a determined content of polyphenols and of proanthocyanidin oligomers, such as the Caophenol® product; the modulating agent capable of enhancing the expression of the aquaporin and/or the number of AQPap channels can be chosen from lycopene encapsulated in the form of nanocapsules, in particular of polycaprolactone comprising a tomato extract at 0.5% enriched with lycopene, aescin, complexes of phospholipids and of horse chestnut bark proanthocyanidins, such as the product sold under the name PA2 Affilene®, cocoa powder extracts, sage extracts, (tridec-2-ynylthio)acetic acid, {[4-(octylthio)but-2-ynyl]thio}acetic acid and retinoids.

The cocoa extracts and encapsulated lycopene as defined above are modulating agents capable of fulfilling both functions. Advantageously, they will be used in the compositions according to the invention, in combination with at least one other slimming agent, in particular at least one other AQPap-modulating agent as defined in the above.

The amount of AQPap modulator present in the composition will be adjusted by a person skilled in the art according to the type of product in order to obtain a cosmetological effective amount, in particular if it is a purified molecule or a plant or cell extract comprising it. By way of indication, the concentration of modulator present in the composition can vary from 0.001 to 20% by weight with respect to the total weight of the composition, in particular from 0.01 to 10%. Thus, compositions suited to the implementation of the invention will comprise, for example, a combination of 0.05 to 0.5% of aescin and of 0.5 to 5% of Dioschol®, without it being possible for these concentrations to be understood as limiting the invention.

Another subject-matter of the invention is the use of at least one compound chosen from Dioscorea opposita sapogenins, such as diosgenin and in particular Dioschol®, lycopene encapsulated in the form of nanocapsules, in particular of polycaprolactone comprising a tomato extract at 0.5% enriched with lycopene, cocoa powder extracts with a determined content of polyphenols and of proanthocyanidin oligomers, aescin, complexes of phospholipids and of horse chestnut bark proanthocyanidins, such as the product sold under the name PA2 Affilene®, cocoa powder extracts, sage extracts, (tridec-2-ynylthio)acetic acid, {[4-(octylthio)but-2-ynyl]thio}acetic acid and retinoids, as agent for increasing the number of the AQPap channels or for stimulating the intrinsic activity of the AQPap channels, as defined in the above, in particular in cosmetic compositions as agent for reducing the volume of the adipocytes and/or for reducing their glycerol content.

According to one of the embodiments of the invention, the AQPap modulator is present in a composition which can additionally comprise at least one lipolytic slimming agent and/or at least one lipogenesis inhibitor slimming agent and/or at least one inhibitor of the adipocyte differenciation slimming agent. The lipolytic and/or slimming action of these agents will be reinforced by the activity of the AQPap modulator, which will facilitate the removal of the glycerol produced by the lipolysis.

The lipolytic agent can be chosen in particular from:
phosphodiesterase inhibitors.

Mention may in particular be made of xanthine bases and the natural extracts comprising them. Examples are composed of caffeine and its derivatives, in particular the 1-hydroxyalkylxanthines disclosed in the document FR-A-2 617 401, caffeine citrate, theophylline and its derivatives, theobromine, acefylline, aminophylline, chloroethyltheophylline, dipropylline, dinipropylline, etamiphylline and its derivatives, etofylline and proxyphylline. These xanthine bases can be used alone or in commercial mixtures, such as the combination of caffeine and of methylsilanetriol alginate and caffeinate sold by Exsymol under the trade name Cafeisilane C. Mention be made, as natural extracts comprising xanthine bases, of tea, coffee, guarana, maté or cola (Cola nitida) extracts and in particular the dry extract of guarana (Paulina sorbilis) fruit comprising from 8 to 10% of caffeine. Mention may also be made of ma huang (ephedra plant) which comprises ephedrine.
α₂-blocking compounds capable of blocking the α₂ receptors at the surface of the adipocytes,
β-adrenergic agonists.

Mention may in particular be made, as β-adrenergic agonist, of alverine or an organic or inorganic salt of alverine, such as alverine citrate.
compounds which inhibit the synthesis of LDL or VLDL receptors,
inhibitors of the enzymes for the synthesis of fatty acids,
   such as acetyl-CoA carboxylase or fatty acid synthetase and in particular cerulenin.
compounds which stimulate β receptors and/or G-proteins,
glucose transportation blockers, such as serine or rutin.
neuropeptide Y (NPY) antagonists capable of blocking the NPY receptors at the surface of the adipocytes,
agents which modify the transportation of fatty acids,
lipolytic peptides and lipolytic proteins,
   such as peptides or proteins, for example the peptides derived from parathyroid hormone, disclosed in particular in Patents FR-2 788 058 and FR-2 781 231, or the peptides disclosed in the document FR-2 786 693, without this list being limited. Mention may also be made of protamines and their derivatives, such as those disclosed in the document FR-2 758 724, and natriuretic peptides.

Other examples of lipolytic agents which can be used are certain plant extracts not comprising caffeine and certain extracts of marine origin. Mention may in particular be made:
as plant extracts: of extracts of Garcinia cambogia, of Bupleurum chinensis, of English ivy (Hedera helix), of arnica (Arnica montana L.), of rosemary (Rosmarinus officinalis N.), of marigold (Calendula officinalis), of sage (Salvia officinalis L.), of ginseng (Panax ginseng), of St John's wort (Hypericum perforatum), of butcher's broom (Ruscus aculeatus L.), of meadowsweet (Filipendula ulmaria L.), of cat's whiskers (Orthosiphon stamineus benth), of birch (Betula alba), of cecropia, of the argania tree, of Ginkgo biloba, of horsetail, of aescin, of cangzhu, of Chrysanthellum indicum, of Disoscoreae rich in diosgenin or hecogenin, including Dioscorea opposita or mexicana or villosa, of plants of the genus Armeniacea, Atractylodis, Platycodon, Sinomenium, Pharbitidis or Flemingia, of Coleus, such as Coleus forskohlii or blumei or esquirolii or scutellaroides or xanthantus or barbatus, such as the Coleus barbatus root extract containing 60% of forskolin, extracts of ballota, of Guioa, of Davallia, of Terminalia, of Barringtonia, of Trema and of Antirobia;
as extracts of marine origin: of algal or phytoplankton extracts, such as an extract of Laminaria digitata sold under the name Phycox75 by Secma, the skeletonema algae, as disclosed in particular in Patent FR-2 782 921, or the Diatomeae, such as those disclosed in Patent FR-2 774 292.

The amount of lipolytic or lipogenesis-inhibiting active agent present in the composition according to the invention can vary to a large extent and will preferably be between 0.001 and 20% by weight, better still between 0.1 and 10% by weight, with respect to the total weight of the composition.

Advantageously, the composition according to the invention comprises at least one diosgenin-rich Dioscorea extract, for example originating from wild yam roots. It is possible, for example, to choose an extract of Discorea opposita roots sold in solution in a derivative of polyethylene glycol (6 EO) and a mixture of mono-, di- and triglycerides of caprylic and capric acids/preservatives/glycerol (ratio by weight 1/93.8/0.2/5), sold under the name Dioschol by Sederma.

According to an advantageous embodiment, the composition additionally comprises at least one agent chosen from desquamating agents, firming agents, agents promoting the synthesis of extracellular matrix constituents, moisturizing agents, aquaporin modulators, agents active with regard to microcirculation, or agents active with regard to the energy metabolism of the cells.

Mention may be made, without implying limitation, of,
active agents which act with regard to microcirculation (vasculoprotective or vasodilatory agents), such as flavonoids, ruscogenins, natural or synthetic esculosides (including Permethol, sold by Sochibo), the aescin extracted from the horse chestnut, nicotinates, hesperidin methyl chalcone, butcher's broom, essential oils of lavender or rosemary, Ammi visnaga extracts, NO donors or direct or indirect modulators of nitric oxide synthase;
firming active agents and/or antiglycant active agents (which prevent sugar from attaching to collagen fibres), such as Centella asiatica and Siegesbeckia extracts, silicon, amadorine, ergothioneine and its derivatives, hydroxystilbenes and their derivatives, in particular resveratrol, plant extracts from the family of the Ericaceae, in particular blueberry (Vaccinium angustifolium) extracts, vitamin C and its derivatives, and retinol and its derivatives.

The term “desquamating agent” is understood to mean any compound capable of acting:
either directly on desquamation by promoting its exfoliation, such as β-hydroxy acids, in particular salicylic acid and its derivatives (including 5-(n-octanoyl)salicylic acid); α-hydroxy acids, such as glycolic acid, citric acid, lactic acid, tartaric acid, malic acid or mandelic acid; urea; gentisic acid; oligofucoses; cinnamic acid; Saphora japonica extract; or resveratrol;
or on enzymes involved in desquamation or decomposition of the corneodesmosomes, glycosidases, stratum corneum chymotryptic enzyme (SCCE) or indeed even other proteases (trypsin, chymotrypsin-like). Mention may be made of chelating agents of inorganic salts: EDTA; N-acyl-N,N′,N′-ethylenediaminetriacetic acid; aminosulphonic compounds and in particular N-(2-hydroxyethyl)piperazine-N′-2-ethanesulphonic acid (HEPES); derivatives of 2-oxothazolidine-4-carboxylic acid (procysteine); derivatives of α-amino acids of glycine type (such as disclosed in EP-0 852 949, and sodium methylglycinediacetate sold by BASF under the tradem Trilon M); honey; or sugar derivatives, such as O-octyanoyl-6-D-maltose and N-acetylglucosamine.

The agents which act with regard to the energetic metabolism of skin cells, such as, for example, and without implying limitation, the synthesis of ATP, those which intervene with regard to the respiratory chain of the cell or with regard to the energy reserves. Mention may be made of coenzyme Q10 (ubiquinone), cytochrome C, creatine or phosphocreatine.

Aquaporin modulators other than AQPap may advantageously be present in the compositions according to the invention, in particular aquaporins involved in water transportation, such as AQP 3 or 4, so as to avoid the formation of oedema and to promote drainage of the adipose tissue. Use is made, for example, of plant extracts comprising such modulators, such as, for example, the extracts cited in WO 0137799, or the cytoskeleton-modifying compounds cited in WO 0164219 from Janssen Pharm, or compounds which can be bonded to lipocalin (WO 0112225, Santen), or α-interferon.

Agents promoting the synthesis of constituents of extra cellular matrix agents are in particular agents stimulating the synthesis of dermal or epidermal macromolecules and/or preventing their degradation.

Among the active agents stimulating the macromolecules of the dermis or preventing their degradation, there may be mentioned those which act:
either on the synthesis of collagen, such as extracts of Centella asiatica; asiaticosides and derivatives; ascorbic acid or vitamin C and its derivatives; synthetic peptides such as iamin, biopeptide CL or palmitoyloligopeptide marketed by the company SEDERMA; peptides extracted from plants, such as the soyabean hydrolysate marketed by the company COLETICA under the trade name Phytokine®; and plant hormones such as auxins and lignans;
or on the synthesis of elastin, such as the extract of Saccharomyces cerevisiae marketed by the company LSN under the trade name Cytovitin®; the extract of the alga Macrocystis pyrifera marketed by the company SECMA under the trade name Kelpadelie®; and N-acylamino-amides compounds such as disclosed in application EP 1 292 608
or on the synthesis of glycosaminoglycans, such as the product of fermentation of milk by Lactobacillus vulgaris, marketed by the company BROOKS under the trade name Biomin yogourth®; the extract of the brown alga Padina pavonica marketed by the company ALBAN MULLER under the trade name HSP3®; the extract of Saccharomyces cerevisiae available in particular from the company SILAB under the trade name Firmalift® or from the company LSN under the trade name Cytovitin®; xylose derivatives such as Aquaxyl®; and C-glycosides compounds such as those disclosed in US-2004-0048785;
or on the synthesis of fibronectin, such as the extract of the zooplankton Salina marketed by the company SEPORGA under the trade name GP4G®; the yeast extract available in particular from the company ALBAN MÜLLER under the trade name Drieline®; and the palmitoyl pentapeptide marketed by the company SEDERMA under the trade name Matrixil®;
or on the inhibition of metalloproteinases (MMP) such as more particularly MMP 1, 2, 3, 9. There may be mentioned: retinoids and derivatives, oligopeptides and lipopeptides, lipoamino acids, the malt extract marketed by the company COLETICA under the trade name Collalift®; extracts of blueberry or of rosemary; lycopene; isoflavones, their derivatives or plant extracts containing them, in particular extracts of soyabean (marketed for example by the company ICHIMARU PHARCOS under the trade name Flavosterone SB®), of red clover, of flax, of kakkon or of sage;
or on the inhibition of serine proteases such as leukocyte elastase or cathepsin G. There may be mentioned: the peptide extract of seeds of a legume (Pisum sativum) marketed by the company LSN under the trade name Parelastyl®; heparinoids; and pseudodipeptides such as {2-[acetyl(3-trifluoromethylphenyl)amino]-3-methylbutyrylamino}acetic acid.

Among the active agents stimulating epidermal macromolecules such as fillagrin and keratins, there may be mentioned in particular the lupin extract marketed by the company SILAB under the trade name Structurine®; the beech Fagus sylvatica bud extract marketed by the company GATTEFOSSE under the trade name Gatuline®; and the zooplankton Salina extract marketed by the company SEPORGA under the trade name GP4G®.

The compositions can also comprise agents promoting sun-tanning, regulating sebum production or photo-protecting agents.

Here again, the amount of these additional active agents can vary to a large extent. Preferably, these active agents will be present in the composition according to the invention in an amount representing from 0.01 to 15% and better still from 0.05 to 10% by weight, with respect to the total weight of the composition.

According to one of the embodiments of the invention, the compositions are essentially devoid of chromium or of its salts, such as chromium picolinate, that is to say that the amounts optionally present are lower than those which make it possible to obtain an effect with regard to slimming.

According to another embodiment, the compositions according to the invention are essentially devoid of carnitine or its derivatives, that is to say that the amounts optionally present are lower than those which make it possible to obtain an effect with regard to slimming and/or lipolysis.

According to one of the embodiments of the invention, the AQPap modulator is used in compositions for external topical use.

It can in particular be in the form of an aqueous, alcoholic, aqueous/alcoholic or oily solution, of a suspension, of a dispersion, of W/O, O/W or multiple emulsions, of aqueous or anhydrous gels, or of vesicular dispersions of ionic or nonionic type. It can have a solid, semi-liquid or pasty consistency.

For topical application, the composition which can be used according to the invention can in particular be in the form of an aqueous, aqueous/alcoholic or oily solution or of a dispersion of the lotion or serum type, of emulsions with a liquid or semi-liquid consistency of the milk type, obtained by dispersion of a fatty phase in an aqueous phase (O/W) or vice versa (W/O), or multiple emulsions, of a free or compacted powder to be used as is or to be incorporated in a physiologically acceptable medium, or of suspensions or emulsions with a soft consistency of the cream or aqueous or anhydrous gel type, or of microcapsules or microparticles, or of the vesicular dispersions of ionic and/or nonionic type. It can thus be provided in the form of an ointment, of a tincture, of a cream, of a balm, of a powder, of a patch, of an impregnated pad, of a solution, of an emulsion or of a dispersion of the vesicular type, of a lotion, of a gel, of a spray, of a suspension, of a shampoo, of an aerosol or of a foam. It can be anhydrous or aqueous. It can also consist of solid preparations constituting cleaning soaps or bars.

These compositions are prepared according to the usual methods.

According to another embodiment of the invention, the composition is suitable for oral use, in particular in “oral cosmetics”.

For use by the oral route, the composition can be provided in particular in the form of capsules, including hard gelatin capsules, of tablets, including sugar-coated tablets, of granules, of chewing gum, of gels or of syrups to be taken orally or in any other form known to a person skilled in the art.

The amounts of the various constituents of the compositions which can be used according to the invention are those conventionally used in the fields under consideration.

The aqueous phase comprises water and optionally an ingredient miscible in any proportion with water, such as C₁ to C₈ alcohols, for example ethanol or isopropanol, polyols, such as propylene glycol, glycerol or sorbitol, or also acetone or ether.

When the composition which can be used according to the invention is an emulsion, the proportion of the fatty phase can range from 2% to 80%, in particular from 5% to 80%, by weight and preferably from 5% to 50% by weight, with respect to the total weight of the composition. The oils, the waxes, the emulsifiers and the coemulsifiers used in the composition in the form of an emulsion are chosen from those conventionally used in the cosmetics field. The emulsifier and the coemulsifier are present in the composition in a proportion ranging from 0.1%, in particular from 0.3% to 30%, by weight, preferably from 0.5% to 20% by weight or better still from 1% to 8%, with respect to the total weight of the composition. The emulsion can additionally comprise lipid vesicles and in particular liposomes.

When the composition which can be used according to the invention is an oily solution or gel, the fatty phase can represent more than 90% of the total weight of the composition.

In a known way, the composition according to the invention can also comprise adjuvants conventional in the cosmetics field, such as hydrophilic or lipophilic gelling or thickening agents, hydrophilic or lipophilic additives, preservatives, antioxidants, solvents, fragrances, fillers, screening agents, odour absorbers or electrolytes, neutralizing agents, UV (irradiation by ultraviolet rays) blocking agents, such as sunscreens, film-forming polymers, cosmetic and pharmaceutical active agents with a beneficial effect on the skin or keratinous fibres, and colouring materials which are soluble or insoluble in the medium. The amounts of these various adjuvants are those conventionally used in the cosmetics field and in particular from 0.01% to 50% of the total weight of the composition, for example from 0.01% to 20%, in particular less than or equal to 10% of the total weight of the composition and especially greater than or equal to 0.1%. These adjuvants, depending on their nature, can be introduced into the fatty phase, into the aqueous phase and/or into lipid spherules, vesicles or microspheres, such as liposomes.

The fatty phase can comprise fatty or oily compounds which are liquid at ambient temperature (25° C.) and atmospheric pressure (760 mm of Hg), generally referred to as oils. These oils may or may not be compatible with one another and may form a macroscopically homogeneous liquid fatty phase or a two- or three-phase system.

The fatty phase can, in addition to the oils, comprise waxes, gums, lipophilic polymers, or “pasty” or viscous products comprising solid parts and liquid parts.

Mention may be made, as oils or waxes which can be used in the invention, of mineral oils (liquid petrolatum, hydrogenated isoparaffin), vegetable oils (liquid fraction of karite butter, sunflower oil, soybean oil, wheat germ oil), animal oils (perhydrosqualene), synthetic oils (purcellin oil, fatty acid esters), silicone oils or waxes (phenyltrimethicone, cyclomethicone, linear or cyclic polydimethylsiloxanes) and fluorinated oils (perfluoropolyethers), beeswax, candelilla wax, carnauba wax or paraffin wax. Free fatty acids (stearic acid, linoleic acid, linolenic acid) and fatty alcohols may be added to these oils and waxes.

Mention may be made, as emulsifiers which can be used in the invention, of, for example, glycerol stearate or laurate, sorbitol stearates or oleates, alkyl dimethicone copolyol (with alkyl≧8) and their mixtures, polyoxyethylenated sorbitol stearate or oleate, for example polysorbate 60 and the PEG-6/PEG-32/Glycol Stearate mixture sold under the name of Tefose® 63 by Gattefossé, polyethylene glycol monostearate or monolaurate, dimethicone copolyols and their mixtures.

Mention may be made, as solvents which can be used in the invention, of lower alcohols, in particular ethanol and isopropanol, or propylene glycol.

Mention may be made, as hydrophilic gelling agents which can be used in the invention, of carboxyvinyl polymers (carbomer), acrylic copolymers, such as acrylate/alkylacrylate copolymers, polyacrylamides, polysaccharides, such as hydroxypropylcellulose, natural gums and clays and mention may be made, as lipophilic gelling agents, of modified clays, such as Bentones®, metal salts of fatty acids, such as aluminium stearates, hydrophobic silica, ethylcellulose and polyethylene.

Advantageously, the composition is provided in the form of a water-in-oil (W/O) emulsion comprising a fatty acid glyceride, an alcohol and a specific silicone surfactant, in particular the dimethicone copolyols sold under the names DC 5329, DC 7439-146 and DC2-5695 by Dow Corning and in particular the mixture of oxyethylenated oxypropylenated (18 EO/18 PO) polydimethylsiloxane, of cyclopentasiloxane and of water (10/88/2 ratio by weight) sold under the name DC2 5225 C by Dow Corning, or KF-6013, KF-6015, KF-6016 and KF-6017 by Shin-Etsu. The silicone emulsifier can be present in the composition according to the invention in an amount of between 0.1% and 5% by weight, with respect to the total weight of the composition, preferably in an amount of between 0.5% and 3% by weight. The silicone emulsifier can be used in combination with a cyclomethicone.

In particular, the compositions comprising Dioschol will comprise an alcohol.

When the modulator is used in a composition for oral use, the latter advantageously comprises, in addition, calcium salts (for example calcium carbonate), calcium originating from dietary dairy products (yogurts, milk, powdered milk, and the like), for example at a dose in the region of 1 g/day (more advantageous), calcium channel modulators, conjugated linoleic acid, direct or indirect NO production modulators, or direct or indirect modulators of nitric oxide synthase. All the derivatives can be used in combination with additional factors, such as leucine or branched amino acids.

Another subject-matter of the invention is the use of at least one agent chosen from aescin, proanthocyanidins, encapsulated lycopene, Caophenol, diosgenin, Dioschol®, ginkgo extracts, sage extracts, (tridec-2-ynylthio)acetic acid, {[4-(octylthio)but-2-ynyl]thio}acetic acid and PA2 Affilene® (or complex of phospholipids and of horse chestnut bark proanthocyanidins 36%) for modulating the aquaglyceroporins of the adipose tissue and thus for reducing the volume of the adipocytes and of the adipose tissue.

According to yet another of its aspects, the invention relates to a cosmetic treatment process intended to prevent or reduce an increase in the volume of adipose tissue and/or the formation of fatty lumps and/or to a slimming process comprising the application, over all or part of the body, of a composition comprising at least one AQPap modulator as defined above. Application can be carried out in particular over areas subject to lipodystrophia, such as the abdomen, the top of the thighs or arms, or some area of the face such as the bottom of the face. This application can advantageously be carried out after a physical effort or in complementing a slimming course of treatment. It can be repeated over time, several times daily and for several weeks or months.

The treatment can also be carried out in complementing a treatment for obesity.

### EXAMPLES

The examples which follow are intended to illustrate the invention.

### Example 1

### Assay of Glycerol Released

The effects on the release of glycerol were tested in the presence of all the test products and using a fluorescence assay method. Glycerol was then assayed with regard to certain selected products using a calorimetric method and in the presence or the absence of HgCl₂.

The majority of mammalian aquaporins are inhibited by mercury and its salts, whereas chemical inhibitors of diffusion are unknown (P. Agre et al., J. of Physiology, 2002, 542.1).

The cells used were mice preadipocytes of the 3T3-L1 line (40 000 cells/well).

The cells were cultured until confluency in a growth medium, with a change in medium every 2-3 days (24-well plates).
Growth medium:
   DMEM (Life Technologies 21969035) L-glutamine, 2 mM (Life Technologies 25030024)
   Penicillin 50 IU/ml streptomycin 50 μg/ml (Life Technologies 15070063)
   Amphotericin B, 0.25 μg/ml (Sigma A2942)
   Foetal calf serum, 10% v/v (Life Technologies 10106151).

At high confluency, the cells were transferred into a differentiation medium for 2×48 hours.
Differentiation medium:
   DMEM (Life Technologies 21969035)
   L-glutamine, 2 mM (Life Technologies 25030024)
   Penicillin 50 IU/ml streptomycin 50 μg/ml (Life Technologies 15070063)
   Foetal calf serum, 10% v/v (Life Technologies 10106151),
   Isobutylmethylxanthine, 0.5 mM (IBMX, Sigma 17018)
   Insulin, 5 μg/ml (Sigma 11882)
   Dexamethasone, 1 μM (Sigma D1756)

After differentiation, the cells were transferred into DMEM medium+insulin (5 μg/ml final) for 72 hours and then into growth medium (DMEM/10% serum).

Control culturings were carried out in 3T3-L1 growth medium throughout the duration of the experiment (undifferentiated 3T3-L1 cells).

Assay of Glycerol Released

1. Fluorescence Assay

Protocol 1 (Without Epinephrine)

The differentiated cells were placed in test medium (MEM 100% (v/v), bicarbonate 1.87 mg/ml, glutamine 2 mM, delipidated bovine serum albumin 0.5% (w/v), penicillin/streptomycin 25 IU/ml/25 μg/ml) and were then treated with the test products or the references. After incubating at 37° C. for 1 h and 5% CO₂, the culture supernatants were removed in order to assay the glycerol released.

The glycerol released into the culture medium was with a determined content of directly by measuring the fluorescence of the NADH produced in the following reaction sequence (according to Wieland). 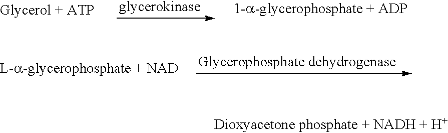
Autofluorescence and quenching (absorption of the fluorescence) controls were carried out in order to determine possible interference of the products in this assay.

Inhibition of the Transportation of Glycol Induced by HgCl₂—Protocol 3

The differentiated cells were placed in test medium for 24 h. The cells were subsequently placed in PBS buffer comprising or not comprising HgCl₂ (30 μM final) for 5 minutes at 37° C. After treatment with HgCl₂, the cells were washed twice in PBS and then placed in test medium comprising or not comprising the test products. The culture supernatants were subsequently removed in order to assay the glycerol released.

2. Colorimetric Assay of Glycerol Released

The release of glycerol by the stimulated cells was, in some cases, confirmed by using a calorimetric test (Roche) according to the instructions of the supplier. This approach allowed us to avoid the problem of autofluorescence of certain products tested, which interferes with the analysis.

Control:

Release of glycerol by undifferentiated 3T3-L1 cells and differentiated 3T3-L1 cells

| Cells | Treatment | Glycerol (μM) | Δ% |
|---|---|---|---|
| Undifferentiated | Unstimulated | 13.5 | 35 |
| preadipocytes | | | |
| Differentiated | Unstimulated | 39.0 | 100 |
| preadipocytes | | | |
| Differentiated | Epinephrine, | 149.5 | 383 |
| preadipocytes | 40 μM | | |

The epinephrine is a positive control for the lipolysis and stimulates the opening of the AQPap channels and the translocation of the AQPap molecules from the cytosol towards the cell membrane.

Products Tested:

Dioschol®

Assay of the Glycerol Released by the Differentiated 3T3-L1 Cells

Protocol 1

| | Glycerol | | | Quenching |
|---|---|---|---|---|
| Treatment | (μM) | sd | % Control | control (%) |
| Control | 33.41 | 5.67 | 100 | 0 |
| Epinephrine | 117.57 | 10.90 | 352 | 0 |
| 40 μM | | | | |
| Dioschol | | | | |
| 0.016% | 126.50 | 7.22 | 346 | 0 |
| 0.0032% | 55.04 | 1.20 | 162 | 0 |
| 0.0006% | 40.77 | 1.44 | 122 | 0 |

Assay of the Glycerol Released by the Differentiated 3T3-L1 Cells

HgCl₂ protocol

| | | |
|---|---|---|
| Treatment | Glycerol (μM) | % Control |

| Epi-free control | | 34.41 | 40 |
|---|---|---|---|
| HgCl₂-free control | | 160.26 | 185 |
| HgCl₂ control | | 86.61 | 100 |
| Dioschol | 0.016% | 127.03 | 106 |
| | 0.0032% | 92.55 | 92 |
| | 0.0006% | 76.65 | 89 |

Encapsulated Lycopene

Colorimetric Assay of the Glycerol Released by the Differentiated 3T3-L1 Cells

Protocol 1

| Treatment | Glycerol (μM) | % Control |
|---|---|---|
| Control | 56.57 | 100 |
| Epinephrine 40 μM | 449.74 | 795 |
| Encapsulated lycopene | | |
| 0.2% | 51.28 | 91 |
| 0.04% | 59.22 | 105 |
| 0.008% | 67.97 | 120 |

Colorimetric Assay of the Glycerol Released by the Differentiated 3T3-L1 Cells

HgCl₂ protocol

| Treatment | Glycerol (μM) | % Control |
|---|---|---|
| Epi-free control | 38.46 | 40 |
| HgCl₂-free control | 413.72 | 432 |
| HgCl₂ control | 95.85 | 100 |
| Encapsulated lycopene | | |
| 0.2% | 94.42 | 99 |
| 0.04% | 91.58 | 96 |
| 0.008% | 86.57 | 89 |

Caophenol®

Assay of the Glycerol Released by the Differentiated 3T3-L1 Cells

Protocol 1

| | | | | % theory, |
|---|---|---|---|---|
| | | | | taking into |
| | | | Quenching | account the |
| | Glycerol | % | control | quenching |
| Treatment | (μM) | Control | (%) | control |
| Control | 31.15 | 100 | 0 | 100 |
| Epinephrine, | 101.85 | 327 | 0 | 327 |
| 40 μM | | | | |
| Caophenol | | | | |
| 2% | 33.31 | 107 | 79 | 510 |
| 0.4% | 39.63 | 127 | 53 | 270 |
| 0.08% | 28.28 | 91 | 22 | 115 |

Assay of the Glycerol Released by the Differentiated 3T3-L1 Cells

HgCl₂ protocol

| | | | | % theory, |
|---|---|---|---|---|
| | | | | taking into |
| | | | Quenching | account the |
| | Glycerol | % | control | quenching |
| Treatment | (μM) | Control | (%) | control |
| Epi-free | 29.71 | 34 | 0 | 34 |
| control | | | | |
| HgCl₂-free | 139.17 | 159 | 0 | 159 |
| control | | | | |
| HgCl₂ control | 87.71 | 100 | 0 | 100 |
| β-Mercaptol, | 135.74 | 155 | 0 | 155 |
| 10 mM | | | | |
| Caophenol | | | | |
| 2% | 30.78 | 35 | 78 | 159 |
| 0.4% | 61.43 | 70 | 63 | 189 |
| 0.08% | 57.62 | 66 | 20 | 100 |

The results are summarized in the following table:

| | Release of |
|---|---|
| Name | glycerol |
| Dioschol 0.0006-0.016% | Yes |
| Mexoryl SAQ ®: Encapsulated | Yes |
| lycopene 0.008-0.2% | |
| Caophenol 0.08-2% | Yes |
| (Tridec-2-ynylthio)acetic acid | No |
| 0.16-4.0 μg/ml | |
| {[4-(Octylthio)but-2-ynyl]thio}acetic acid | No |
| 0.32-8.0 μg/ml | |
| Ivy 0.016-0.4% | No |

Ivy, known as a slimming agent, is not active with regard to the AQPap channels.

A release of glycerol stimulated by Dioschol, encapsulated lycopene and Caophenol is observed. In the presence of mercuric chloride, which blocks the AQPap channels, no departure of glycerol is observed, even in the presence of the active agents. This demonstrates that the release of glycerol observed passes through the adipocyte aquaglyceroporin channels.

### Example 2

### Measurement of the Increase in the Relative Expression of AQPap

The test was carried out on differentiated 3T3-L1 cells obtained as described in Example 1.

The differentiated cells were placed beforehand in test medium for 24 hours and were then treated with the test products or the references. After incubating for 6 h at 37° C. and 5% CO₂, the culture supernatants were removed, the cells were rinsed with a PBS solution and the cell layers were placed in Tri-Reagent (Sigma T9424) and then immediately frozen at −80° C.

The expression of the markers selected was evaluated by RT-Q-PCR with regard to the messenger RNAs extracted from the cells corresponding to each treatment.

The marker β-actin (M12481) was used as reference marker.

The operational stages are as follows:
Extraction of the total RNAs using Tri-Reagent according to the protocol recommended by the supplier. Then further extraction with chloroform and precipitation with isopropanol.
Removal of the traces of potentially contaminating DNA by treatment with the DNA-free system (Ambion). After monitoring the quality of the RNAs obtained, the three samples corresponding to each treatment were pooled.
Carrying out the reverse transcription reaction on the mRNA in the presence of the oligo(dT) primer and of the Superscript II (Gibco) enzyme.
Quantification by fluorescence of the synthesized cDNA and adjustment of the concentrations to 50 ng/μl. Further quantification of each cDNA, after final dilution, is carried out before the PCR reaction.

Quantitative PCR

The PCR (polymerase chain reactions) reactions were carried out by quantitative PCR with the “Light Cycler” system (Roche Molecular Systems Inc.) and according to the procedures recommended by the supplier.

Analysis of the Q-PCRs

The incorporation of fluorescence into the amplified DNA is carried out continuously during the PCR cycles. This system makes it possible to obtain curves of measurement of the fluorescence as a function of the PCR cycles and to thus evaluate a value of relative expression for each marker.

The number of cycles is determined from the “exit” points of the fluorescence curves. For the same marker analysed, the later the sample exits (high cycle number), the lower the initial number of mRNA copies.

The RE (relative expression) value is expressed in arbitrary units according to the following formula:
(1/2^{number of cycles})×10⁶

Results: Effects of the various treatments on the relative expression of the Aquaporin adipose (AQPap) messenger. Results with respect to the amount of actin messenger (reference gene).

RE=Relative Expression, expressed in arbitrary units (AU), n=2.

AQP7/Actin

| | | | RE*AQP7 | % |
|---|---|---|---|---|
| Treatment | Concentration | | (AU) | Control |
| Undifferentiated | — | | 0.0011 | 3 |
| control | | | | |
| Differentiated | — | | 0.048 | 100 |
| control | | | | |
| Aescin | 0.0032 | mg/ml | 0.043 | 164 |
| | 0.00064 | mg/ml | 0.039 | 168 |
| | 0.000128 | mg/ml | 0.034 | 134 |
| PA2 Affilene | 0.01 | mg/ml | 0.026 | 233 |
| | 0.02 | mg/ml | 0.051 | 201 |
| | 0.004 | mg/ml | 0.051 | 145 |
| Encapsulated | 0.02% | | 0.025 | 147 |
| lycopene | 0.04% | | 0.024 | 177 |
| | 0.008% | | 0.022 | 139 |
| (Tridec-2- | 0.008 | mg/ml | 0.032 | 174 |
| ynylthio)acetic | 0.0016 | mg/ml | 0.872 | 105 |
| acid | 0.00032 | mg/ml | 0.912 | 130 |
| Fermented sage | 0.04 | mg/ml | 0.020 | 175 |
| compound (FSC) | 0.008 | mg/ml | 0.020 | 155 |
| | 0.0016 | mg/ml | 0.025 | 159 |
| {[4-(Octylthio)- | 0.004 | mg/ml | 0.020 | 167 |
| but-2- | 0.0008 | mg/ml | 0.022 | 153 |
| ynyl]thio}acetic | 0.00016 | mg/ml | 0.023 | 128 |
| acid | | | | |

The results are summarized in the following table:

| | mRNA |
|---|---|
| Name | stimulation |
| Dioschol ® 0.0006-0.016% | No |
| Encapsulated lycopene 0.008-0.2% | Yes |
| Caophenol 0.08-2% | No |
| Aescin (α₂ block.) 0.128-3.2 μg/ml | Yes |
| PA2 Affilene 0.004-0.1 mg/ml | Yes |
| Fermented sage compound (FSC) | Yes |
| 1.6-40.0 μg/ml | |
| (Tridec-2-ynylthio)acetic acid | Yes |
| 0.16-4.0 μg/ml | |
| {[4-(Octylthio)but-2-ynyl]thio}acetic acid | Yes |
| 0.32-8.0 μg/ml | |
| Ivy 0.016-0.4% | No |

### Example 3

### Composition for Topical Use

A water/oil emulsion is prepared with the following formulation:

| Aqueous phase | |
|---|---|
| Water | q.s for 100 |
| Aescin | 0.45 |
| Mg sulphate | 0.7 |
| Glycerol | 12 |
| Butylene glycol | 9 |
| Dioschol (1) | 3 |
| Thermal water | 5 |
| Ethanol | 20 |
| Oily phase | |
| Cyclopentasiloxane | 10 |
| Apricot oil | 10 |
| Fragrance | 0.3 |
| DC2-5225C (2) | 10 |

(1) Dioschol: Extract of Dioscorea opposita (wild yam) roots in a mixture: derivative of polyethylene glycol (6 EO) and of mixture of mono-, di- and triglycerides of caprylic and capric acids/preservatives/glycerol (1/93.8/0.2/5 ratio by weight) sold by Sederma.
(2) DC2 5225 C: mixture of oxyethylenated oxypropylenated (18 EO/18 PO) polydimethylsiloxane, of cyclopentasiloxane and of water (10/88/2 ratio by weight) sold by Dow Corning.

### Example 4

### Slimming Composition for Topical Use

| Aqueous phase | |
|---|---|
| Water | q.s. for 100 |
| Caffeine | 3 |
| Aescin | 0.2 |
| Salicylic acid | 0.72 |
| Mg sulphate | 0.7 |
| Trisodium citrate | 2 |
| Glycerol | 8 |
| Butylene glycol | 5 |
| Dioschol (1) | 3 |
| Thermal water | 5 |
| Ethanol | 20 |
| Preservatives | 0.5 |
| Colorants | 0.0001 |
| Neutralizing agent | 0.72 |
| Oily Phase | |
| Cyclopentasiloxane | 9 |
| Isoparaffin | 2 |
| Cyclohexasiloxane | 5 |
| Fragrance | 0.3 |
| DC2-5225C (2) | 8 |

(1) Dioschol: Extract of Dioscorea opposita (wild yam) roots in a mixture: derivative of polyethylene glycol (6 EO) and of mixture of mono-, di- and triglycerides of caprylic and capric acids/preservatives/glycerol (1/93.8/0.2/5 ratio by weight) sold by Sederma.
(2) DC2 5225 C: mixture of oxyethylenated oxypropylenated (18 EO/18 PO) polydimethylsiloxane, of cyclopentasiloxane and of water (10/88/2 ratio by weight) sold by Dow Corning.

The compositions of Examples 3 and 4 are prepared in a way conventional to a person skilled in the art.

The aqueous phase and the oily phase are prepared separately under cold conditions. The aqueous phase is subsequently dispersed with vigorous stirring in the oily phase.

All publications cited in the specification, both patent publications and non-patent publications, are indicative of the level of skill of those skilled in the art to which this invention pertains. All these publications are herein incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A method for reducing the volume of adipocytes, comprising:
administering to a human a cosmetic composition comprising at least one aquaglyceroporin adipose (AQPap) modulator in an amount effective to reduce volume of adipocytes.

2. The method of claim 1, wherein said cosmetic composition comprises two AQPap modulators, wherein a first modulator enhances synthesis of aquaglyceroporin adipose by the adipocytes and a second modulator stimulates the intrinsic activity of the AQPap channels of adipocytes.

3. The method of claim 1, wherein said AQPap modulator enhances departure of glycerol from adipocytes.

4. The method of claim 1, wherein said AQPap modulator promotes the opening of AQPap channels or promotes their translocation towards a membrane.

5. The method of claim 1, wherein said at least one AQPap modulator is selected from the group consisting of polyphenols, procyanidins, catechins in the monomer or polymer forms, anthocyanidins, proanthocyanidins, flavonoids, sapogenins, pycnogenol, phyto-oestrogens, quercetin, (tridec-2-ynylthio)acetic acid, {[4-(octylthio)but-2-ynyl]thio}acetic acid, plant extracts comprising the same, retinoids and plant extracts chosen from ginseng, sage, chocolate, cocoa, apples, red wine, bilberries, blueberry, Dioscorea opposita, the herb Hypericum perforatum, Hamamelis, Ceratonia siliqua (locust bean), Musanga cecropioides, Crataegus laevigata (hawthorn), coconut (Cocos nucifer) fibres, Sorgum bicolour, Pyrus communis, kiwi fruit, tomato, aubergine, Guazuma ulmifolia, rhubarb, Uncaria sp., and their mixtures.

6. The method of claim 1, wherein the procyanidins comprise procyanidin B2 or procyanidin C1.

7. The method of claim 1, wherein said at least one AQPap modulator is selected from the group consisting of cocoa powder extracts with a determined content of polyphenols, proanthocyanidin oligomers, aescin, horse chestnut proanthocyanidins, complexes of phospholipids, complexes of horse chestnut bark proanthocyanidins, and extracts of Dioscorea opposita tuber with a determined content of diosgenin.

8. The method of claim 1, wherein said at least one AQPap modulator comprises at least (i) one modulator which increases the intrinsic activity of the AQPap selected from the group consisting of cocoa powder extracts, Dioscorea opposita sapogenins and lycopene encapsulated in the form of nanocapsules, and (ii) one modulator which enhances the synthesis of AQPap selected from the group consisting of aescin, complexes of phospholipids and of horse chestnut bark proanthocyanidins, and retinoids.

9. The method of claim 1, wherein said at least one AQPap modulator, when introduced into a culture of mature adipocytes, induces an increase in the synthesis of AQPap by these adipocytes of greater than or equal to 1.5 times, with respect to a culture of mature adipocytes in the absence of AQPap modulator.

10. The method of claim 1, wherein said at least one AQPap modulator increases by at least 1.2 times the amount of glycerol released into the culture supernatant of mature adipocytes, with respect to the amount of glycerol in the culture supernatant of mature adipocytes which have not received AQPap modulator.

11. The method of claim 1, wherein said cosmetic composition further comprises at least one lipolytic inhibitor, lipogenesis inhibitor, or an adipocyte differentiation inhibitor slimming agent, and mixtures of two or more thereof.

12. The method of claim 11, wherein said at least one lipolytic inhibitor is selected from the group consisting of phosphodiesterase inhibitors, inhibitors of α₂ receptors, inhibitors of NPY receptors, inhibitors of the synthesis of LDL or VLDL receptors, activators of β receptors, activators of G-proteins, glucose transportation blockers, adenylate cyclase activators, lipolytic peptides and lipolytic proteins.

13. The method of claim 1, wherein said AQPap modulator is present in said cosmetic composition in amount about 0.001% to about 20%, based on the total weight of said composition.

14. The method of claim 1, wherein said cosmetic composition is administered topically.

15. The method of claim 1, wherein said cosmetic composition further comprises at least one agent selected from the group consisting of desquamating agents, firming agents, moisturizing agents, aquaporin modulators, agents active with regard to microcirculation, and agents active with regard to the energy metabolism of the cells.

16. The method of claim 1, wherein said cosmetic composition is in the form of an aqueous, alcoholic, aqueous/alcoholic or oily solution, of a suspension, of a dispersion, of W/O, O/W or multiple emulsions, of aqueous or anhydrous gels or of vesicular dispersions of ionic or nonionic type.

17. The method of claim 1, wherein said cosmetic composition is administered orally.

18. The method of claim 1, wherein said cosmetic composition further comprises at least one compound selected from the group consisting of calcium salts, calcium channel modulators, conjugated linoleic acid, NO production modulators, direct or indirect modulators of nitric oxide synthase, leucine and branched amino acids.

19. The method of claim 1, wherein said cosmetic composition further comprises at least one agent selected from the group consisting of aescin, proanthocyanidins, diosgenin and gingko extracts that modulate aquaglyceroporins of adipose tissue.

20. A cosmetic composition comprising;
at least one agent that modulates aquaglyceroporin adipose (AQPap); and
at least one lipolytic active agent.

21. A cosmetic composition for reducing the volume of adipocytes, comprising:
a first AQPap modulator that enhances synthesis of aquaglyceroporin adipose by the adipocytes; and
a second AQPap modulator that stimulates the intrinsic activity of the AQPap channels of adipocytes.
